(19) United States
(12) Patent Application Publication
(10) Pub. No.: US 2008/0022757 A1
(43) Pub. Date: Jan. 31, 2008

(54) Oxidation stability measurement for oil condition management

(75) Inventors: Ji Cang Zhou, Shanghai (CN); Weijun Wang, Shanghai (CN); Maosong Wu, Shanghai (CN)

Correspondence Address: Attorney, Intellectual Property;Honeywell International Inc., 101 Columbia Rd., P.O. Box 2245, Morristown, NJ, 07962, US

(73) Assignee: Honeywell International Inc.

(21) Appl. No.: 11/495,094

(22) Filed: Jul. 27, 2006

(51) Int. Cl. G01N 33/26

(52) U.S. Cl. 73/53.05

(57) ABSTRACT

A sensor apparatus is disclosed that includes a testing chamber or testing chamber arrays configured to receive a sample of oil to be tested. A first embedded heater is coupled to the testing chamber or testing chamber arrays and configured to supply heat to the sample. An oxygen flux chamber is coupled to the testing chamber or testing chamber arrays and configured to provide a substantially stable oxygen flux to the testing chamber. A control module is coupled to the testing chamber, the first embedded heater, and the oxygen flux chamber. The control module is configured to monitor the temperature of the sample, direct the first embedded heater to supply heat to the sample, direct the oxygen flux chamber to provide oxygen flux to the testing chamber, measure an oxygen induction time (OIT), and evaluate the oxidation stability of the sample based on the OIT. A method for thermal analysis of oil oxidation stability is also disclosed.

### TECHNICAL FIELD

Embodiments are generally related to sensing devices and components thereof. Embodiments are also related to oil quality sensors. Embodiments are also related to microheaters and microheater arrays. Embodiments are additionally related to oil oxidation stability measurement systems and methods.

### BACKGROUND OF THE INVENTION

Natural and synthetic lubricating oils are employed in a wide array of modern devices and systems. For example, the modern Diesel and standard internal combustion engines use lubricating oils to improve engine performance, clean engine parts, and reduce friction and heat between moving parts. Cooking oils are also used in food preparation, and in some circumstances form part of a large-scale mass-production system. Monitoring the quality and quantity of useful oils has long been a concern in the industries that employ these oils.

In many modern systems, oil quality sensors are used in an integrated system for monitoring certain oil qualities that aim, for example, to protect an engine in a proactive way. For engine lubricating oil, there are generally three main groups of qualities or properties that are typically monitored.

First, the viscosity and/or lubricity of the base oil itself are useful measurements. Second, monitoring the additives employed to control contaminants is also important. Typically, oil additives include anti-oxidants to help keep the oil from breaking down, pH control additives to help neutralize acidic components that corrode metallic surfaces, and detergents that help keep particulates suspended. One skilled in the art will understand that other additives are also employed. Third, undesirable matter is also monitored. Typically, undesirable matter includes solids, such as “soot” or fine carbon particles, large “agglomerated” carbon particles, and metal particles. Generally, the more important measurements seem to be: viscosity, pH (or etch rate), large particles, and metal particles.

As such, a variety of oil parameters can be monitored, including viscosity, TAN, TBN, particulates, contaminations, and other suitable parameters. There are four parameters in particular that are often specially monitored, including viscosity, TAN, TBN, and oil oxidation stability. Generally, as used herein, “TAN” refers to the Total Acid Number (TAN), and is a measure of acid build-up in the oil. Generally, as used herein, “TBN” refers to the Total Base Number (TBN), and is a measure of the reserve alkalinity in engine oils. One skilled in the art will understand that because acids form in the oil (e.g., sulfates and nitrates), lubricants are often manufactured with a high alkaline count to counteract the acids.

Over time, a lubricant's essential properties may deteriorate through a number of ways, including additive depletion, base oil oxidation, and/or contamination. Generally, oxidation is a chemical change that prevents the oil from performing its job. Oxidation naturally results when a lubricant is repeatedly heated up and cooled down. Appropriate change intervals can help prevent oxidation from becoming a fatal problem, however, oxidation monitoring is usually necessary to identify what change interval is appropriate.

Generally, sulfonation/nitration occurs as a portion of the engine exhaust gets ingested back into the crankcase and the lubricant bonds with the gases, forming sulfates (sulfur compounds) and nitrates (nitrogen compounds) in the oil. These compounds attack metal surfaces and cause metal corrosion. As described above, the TBN is a measure of the reserve alkalinity in engine oils. Because acids form in the oil (sulfates and nitrates), lubricants are manufactured with a high alkaline count (TBN) to counteract the acids. Similarly, the TAN is a measure of the acid build-up in the oil.

Contamination also deteriorates a lubricant oil's essential properties. For example, while soot in the oil is a natural occurrence for diesel engines, too much soot causes the lubricant to become too viscous and thus not lubricate well. Also, soot can build up or group together and cause significant deposits. For this reason, additives such as calcium are added to some lubricants to prevent this build-up. Other common contaminants include, for example, water, glycerol, and fuel dilution. One skilled in the art will understand that a thorough lubricant analysis often includes a “wear debris analysis (WDA).” In order to simplify the following discussion, WDA-related topics are omitted except where necessary to understand one or more embodiments.

There are a number of factors that contribute to an oil's oxidation value. For example, detergents in oil clean surfaces, in part, by inhibiting soot deposition. Detergent additives also help chemically protect oil from attack, and neutralize acids that are formed by fuel combustion. Dispersants are chemicals that assist the detergent additive to keep the contamination in stable suspension form. Both detergents and additives contribute to the lubricant's oxidation value.

Representative detergents include: synthetic sulfonate, potassium salicylate (which particularly effects TAN/TBN), normal phenate, phosphonate, and thiopyrophosphonate. Representative dispersants include succinimide-type dispersants, which particularly affect an oil's TAN/TBN. One skilled in the art will recognize other suitable representative detergents and dispersants.

Corrosion inhibitors and anti-oxidants also contribute to an oil's oxidation value. Corrosion inhibitors help prevent surface corrosion by covering exposed surfaces with a layer of additive. Corrosion inhibitors also react preferentially with air, thus inhibiting the air's attack on metal surfaces. Anti-oxidants in a lubricant protect the lubricant from oxidation and are designed to be sacrificially consumed. When the anti-oxidant and corrosion inhibitor levels have been depleted to approximately 30% of their original value, degradation of the lubricant can occur, indicating that the oil should be exchanged for new oil. A representative corrosion inhibitor is zinc dithiophosphate and a representative anti-oxidant is 2,6-di-tert-butyl para cresol. One skilled in the art will recognize other suitable representative corrosion inhibitors and anti-oxidants.

Over the development of oil monitoring technologies, great efforts have been put into technologies focused on measuring/monitoring oil viscosity, TAN, and TBN. As a result, less effort has been directed towards oil oxidation stability monitoring. Currently, oil oxidation stability measurements are typically carried out using bench-top thermal analysis instruments such as modulated TGA (thermogravimetric analysis, MTGA) and/or pressure DSC (differential scanning calorimetry, PDSC). However, such instruments are not suitable for real-time measurements.

Therefore, what is required is a system, apparatus, and/or method that provides an improved oxidation stability measurement for oil condition management that overcomes at least some of the limitations of previous systems and/or methods.

### BRIEF SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is, therefore, one aspect of the present invention to provide for an improved sensing device.

It is a further aspect of the present invention to provide for an improved oil quality sensor and/or sensor array.

It is a further aspect of the present invention to provide for an improved microheater and microheater array.

It is a further aspect of the present invention to provide for an improved oil oxidation time measurement device.

It is a further aspect of the present invention to provide for an improved oxygen induction time measurement system and method.

The aforementioned aspects and other objectives and advantages can now be achieved as described herein. In a first configuration, a sensor apparatus is disclosed that includes a testing chamber configured to receive a sample of oil to be tested. A first embedded heater is coupled to the testing chamber and configured to supply heat to the sample. An oxygen flux chamber is coupled to the testing chamber and configured to provide a substantially stable oxygen flux to the testing chamber. A control module is coupled to the testing chamber, the first embedded heater, and the oxygen flux chamber. The control module is configured to monitor the temperature of the sample, direct the first embedded heater to supply heat to the sample, direct the oxygen flux chamber to provide oxygen flux to the testing chamber, measure an oxygen induction time (OIT), and evaluate the oxidation stability of the sample based on the OIT.

In a second configuration, a sensor apparatus is disclosed that includes an array of testing chambers configured to receive oil samples to be tested. One of a plurality of embedded heaters is coupled to each of the testing chambers, and each embedded heater is configured to supply heat to the samples. An oxygen flux chamber is coupled to each of the testing chambers and configured to provide a substantially stable oxygen flux to the testing chambers. A control module is coupled to the testing chambers, the plurality of embedded heaters, and the oxygen flux chamber. The control module is configured to monitor the temperature of the samples, direct the plurality of embedded heaters to supply heat to the samples, direct the oxygen flux chamber to provide oxygen flux to the testing chambers, measure oxygen induction time (OIT), and evaluate the oxidation stability of the samples based on the OIT. A method for thermal analysis of oil oxidation stability is also disclosed.

### BRIEF DESCRIPTION OF THE DRAWINGS

The accompanying figures, in which like reference numerals refer to identical or functionally-similar elements throughout the separate views and which are incorporated in and form a part of the specification, further illustrate the embodiments and, together with the detailed description, serve to explain the embodiments disclosed herein.

FIG. 1 illustrates a high-level oil quality management system, in accordance with a preferred embodiment;

FIG. 2 illustrates an oil quality management system, in accordance with a preferred embodiment;

FIG. 2A illustrates an oil quality management system, in accordance with a preferred embodiment; and

FIG. 3 illustrates a flow diagram depicting logical operational steps of a method for thermal analysis of oil oxidation stability, in accordance with a preferred embodiment.

### DETAILED DESCRIPTION

The particular values and configurations discussed in these non-limiting examples can be varied and are cited merely to illustrate at least one embodiment and are not intended to limit the scope thereof.

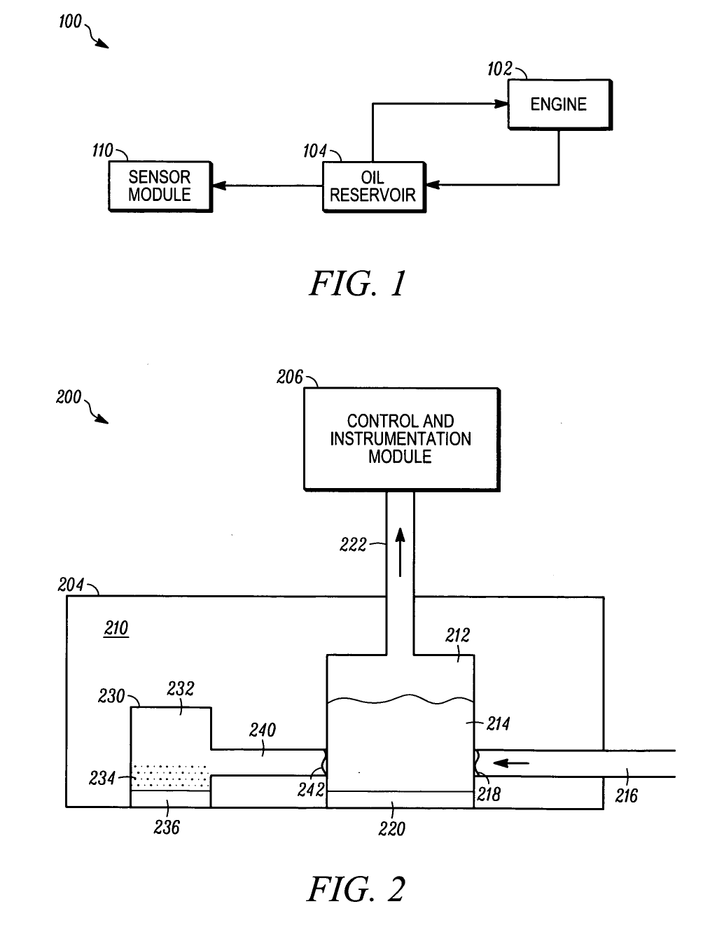

FIG. 1 illustrates a high-level oil quality management system in accordance with one embodiment of the present invention. In particular, FIG. 1 is a block diagram illustrating a sensing system, generally indicated by reference numeral 100. As described in additional detail below, the present invention allows for a means of quantifying in-use oil quality, in particular oil oxidation induction time (OIT), with improved performance over current devices on the market, which, in turn, allows for an improvement in system-wide oil quality and replacement management.

For ease of illustration, the embodiments disclosed herein are described with respect to motor/engine lubricating. One skilled in the art will understand that the embodiments disclosed herein can also be employed to manage the condition of other oil products in other contexts, in some cases with minor or no modification. For example, the systems and/or methods disclosed herein can be applied equally well to cooking oil, food oil, or other oil, whether edible or not.

In the illustrated embodiment, FIG. 1 depicts a high-level block diagram of a sensing system 100. System 100 includes an engine 102, an oil reservoir 104, and a sensor module 110. Generally, engine 102 is an otherwise conventional engine, lubricated at least in part by lubricating oil from oil reservoir 104. Generally, oil reservoir 104 is an otherwise conventional oil reservoir. For ease of illustration, various pumps, valves, connectors and other components employed by oil reservoir 104 and/or engine 102 to move oil to and recover oil from the engine 102 have been omitted insofar as they are not required for an understanding of the present invention.

In the illustrated embodiment, sensor module 110 couples to oil reservoir 104 and is described in more detail below. In an alternate embodiment, sensor module 110 is coupled to engine 102. One skilled in the art will understand that other configurations can also be employed.

Generally, system 100 can be configured to conform to proactive and/or predictive maintenance standards and/or goals. Broadly, predictive maintenance targets detection of incipient failure of both the fluids' properties and certain machine components (e.g., bearings, gears, etc.). Predictive maintenance looks for failure symptoms and faults, employing technologies such as, for example, wear debris analysis, vibration analysis, thermography, and motor current analysis. Predictive maintenance is typically directed towards early detection of faults and/or failure.

Generally, proactive maintenance involves continuous monitoring and controlling of machine failure root causes. Proactive maintenance looks for root causes, employing technologies such as, for example, contaminant monitoring, balancing and alignment tools, viscosity and “AN” monitoring. Typical implementations of proactive maintenance involve three broad steps or stages. In a first step or stage, a target, or standard, associated with each root cause is identified. In a second step or stage, the fluids' conditions are maintained within these targets. In a third step or stage, a feedback loop of an oil analysis program is provided. Proactive maintenance is typically directed towards fault free machines and machine life extension.

There is no single marker typically employed that independently identifies when an oil is no longer usable. Modern oil analysis labs generally observe a combination of markers to determine oil degradation status, but the importance assigned to individual markers, as well as their limits, varies in industrial practices. Many oil analysis labs use the following physical/chemical markers for oil deterioration:

TAN limits: 4-7 mg KOH/g sample
TBN limits: 1-2 mg KOH/g sample
Viscosity change limits: 20-50%
DSC limits: 2-5 minutes (onset time of oxidation).

### One skilled in the art will understand that other suitable physical/chemical markers can also be employed.

Further, there are a number of oil acidity (i.e., pH) related measurements. Generally oil deterioration monitoring can include base oil oxidation monitoring, anti-oxidant and corrosion inhibitors degradation monitoring, and/or detergent/dispersant degradation monitoring. Further, oil acidity can be measured directly, through a variety of pH tests, or indirectly, through a variety of TAN/TBN measurements. Additionally, corrosion/etch rate can be measured through application to sacrificial metals.

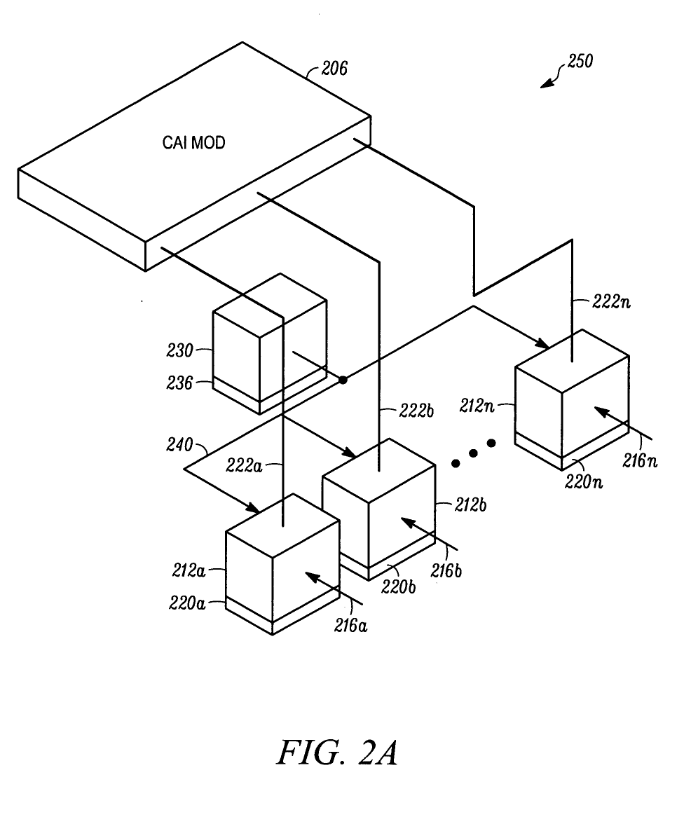

In the illustrated embodiments, sensor module 110 (and subsequent embodiments) is configured to measure oxidation stability. In particular, illustrated embodiments are configured to measure oxygen stability through evaluation of an oxygen induction time (OIT). In order to illustrate certain details of the sensor module 110 embodied in the above exemplary embodiment, reference is now made to FIG. 2.

In particular, FIG. 2 is a block diagram illustrating an oil quality monitoring system, generally designated by the reference numeral 200. In the illustrated embodiment, system 200 is shown with a single testing chamber, as described in more detail below. So configured, system can measure one oil sample at a time. In an alternate embodiment, described below in conjunction with FIG. 2A, the system is shown with an array of testing chambers that can measure more than one oil sample at a time. In the embodiment illustrated in FIG. 2, system 200 includes a test array 204 coupled to a control and instrumentation (“CAI”) module 206. CAI module 206 comprises a variety of control circuitry and measurement circuitry/devices. In general, CAI module 206 controls actions of test array 204 and performs physical measurements on samples prepared for testing by test array 204, as described in more detail below. In a preferred embodiment, CAI module 206 includes electronics for control of heating and cooling profiles of embedded microheaters of test array 204 and a data management system for data acquisition and analysis.

In one embodiment, test array 204 is configured as a disposable module. So configured, test array 204 can be constructed as a replaceable cartridge. Additionally, so configured, system 200 can be adapted for portable use. This embodiment is preferred for applications wherein the oil to be tested does not require repeated real-time monitoring, such as, for example, food oil testing, or wherein a plurality of oils are to be tested, in a variety of physical locations. In an alternate embodiment, test array 204 is configured as a non-disposable module, permanently coupled to CAI module 206. This embodiment is preferred for applications wherein it is desired that the oil to be tested be measured frequently, in real-time, or otherwise repeatedly. One skilled in the art will understand that other configurations can also be employed.

Test array 204 includes housing chamber 210. Housing chamber 210 can be configured to conform to any number of suitable shapes, including custom shapes, to suit the broader system in which system 200 is employed. Housing chamber 210 includes and envelops testing chamber 212. Generally, testing chamber 212 receives an oil sample to be tested (oil 214) through an inlet 216. Testing chamber 212 couples to CAI module 206 through outlet 222.

In the illustrated embodiment, inlet 216 couples to testing chamber 212 through one-way seal 218. Inlet 216 is configured to receive oil 214 from, for example, an oil reservoir 104 of FIG. 1. In the illustrated embodiment, one-way seal 218 and inlet 216 are together configured to deliver a particular quantity of oil 214 to testing chamber 212. In one embodiment, the sample size of oil 214 is in the range of 0.5 to 50 μl, and a range of 5 to 15 μl is preferred. In a preferred embodiment, inlet 216 and one-way seal 218 together comprise an oil sampling probe configured to take a sample size of oil into testing chamber 212.

Oil 214 is held in testing chamber 212 and heated by first heater 220. In one embodiment, first heater 220 comprises a heater and a temperature sensor. In a preferred embodiment, first heater 220 is a microheater. In an alternate embodiment, first heater 220 is a microbrick. In an alternate embodiment, first heater 220 is a microbridge.

Housing chamber 210 includes reagent module 230. Reagent module 230 includes reagent chamber 232, which holds reagent 234. In one embodiment, reagent chamber 232 is an oxygen flux chamber, described in more detail below. Reagent 234 is held in reagent chamber 232 and heated by second heater 236. In a preferred embodiment, second heater 236 is a microheater. In an alternate embodiment, second heater 236 is a microbrick. In an alternate embodiment, second heater 236 is a microbridge.

Reagent module 230 couples to testing chamber 212 through port 240 and one-way seal 242. In the illustrated embodiment, one-way seal 242 and port 240 are together configured to deliver a particular quantity of oxygen flux to testing chamber 212, as described in more detail below. In a preferred embodiment, port 240 and one-way seal 242 together comprise an oxygen flux probe configured to deliver a regulated amount of oxygen flux into testing chamber 212.

Generally, reagent 234 is a single substance or a group (whether composite or otherwise) of substances that release oxygen. In the illustrated embodiment, reagent 234 is configured to release oxygen when heated. In a preferred embodiment, reagent 234 is configured to release a stable and predictable amount of oxygen, or oxygen flux, under constant environmental conditions. In one embodiment, reagent 234 is any type of oxygen source that can release oxygen upon heating. Reagent 234 may be one or more of any number of substances, including, for example, inorganic superoxide, chlorate, and/or perclorate, as well as inorganic or organic Ozonide or other suitable substances. One skilled in the art will understand that a variety of oxygen releasing substances can be employed. In particular, the following are suitable reagents 234: inorganic compounds such as KMnO4, La(NO3)3, CeO2, MgO2 and organic compounds such as benzoyl peroxide (BPO). In a preferred embodiment, reagent 234 is KMnO4.

Generally, in operation, system 200 is configured as follows. A sample of oil 214 is added to testing chamber 212 through inlet 216 and one-way seal 218. Testing chamber 212 is heated to and kept at a stable temperature by first heater 220. In one embodiment, the stable temperature is 150 degrees Celsius. In an alternate embodiment, the stable temperature is 80 degrees Celsius. In an alternate embodiment, testing chamber 212 is heated to different temperatures in a measurement panel, such as 80, 100, 125 and 150 degrees Celsius. One skilled in the art will understand that other configurations, particularly other specific temperatures, can also be employed.

Reagent chamber 232 is heated by second heater 236, causing reagent 234 to produce stable oxygen flux, which is delivered to testing chamber 212 through port 240 and one-way seal 242. The oxidation induction time (OIT) is measured. In one embodiment, the OIT starts from the point that the oxygen releasing reagents 234 were heated and ends upon the point that the temperature sensor in first heater 220 detects a specified value of temperature increase in testing chamber 212. In one embodiment, the temperature increase can be measured as a temperature increase in the oil 214.

Thus, in one embodiment, the OIT is calculated from the first introduction of oxygen flux into testing chamber 212 from reagent chamber 232, to the detection of a pre-determined increase in temperature of the testing chamber 212. In one embodiment, the first introduction of oxygen flux is set at the point where heat is applied by second heater 236 to reagent chamber 232. One skilled in the art will understand that as the first heater 220 is kept at stable operating parameters once the testing chamber 212 reaches the initial stable temperature, the increase in temperature in testing chamber 212 is a function of the stable oxygen flux entering testing chamber 212 from reagent chamber 232, and thus is a function of the oil oxidation induction. The measured OIT is then used to calculate the oxidation stability of the sample oil 214. As described above, the oxidation stability of an oil sample can be employed as a measure of the quality of the oil sample. Thus, generally, in one embodiment, system 200 provides an improved system for measuring oil quality as a function of oxidation induction time.

Referring now to FIG. 2A, an alternate embodiment is depicted in a block diagram illustrating an oil quality monitoring system, generally designated by the reference numeral 250. In particular, system 250 is shown with an array of testing chambers comprising a plurality of testing chambers, 212a, 212b, and so forth, to 212n. Each testing chamber is coupled to an embedded heater 220a, 220b, and so forth, to 220n, respectively. Each testing chamber receives an oil sample through an associated inlet 216a, 216b, and so forth, to 216n, respectively. Each testing chamber is also coupled to CAI module 206 through an independent conduit 222a, 222b, and so forth to 222n. Further, reagent module 230 couples to each testing chamber though port 240. So configured, system 250 can simultaneously measure the oxidation induction time of more than one sample. Specifically, system 250 is configured to measure the oxidation induction time of n samples, where n is the number of testing chambers in the array.

Additionally, in the illustrated embodiment, system 250 is depicted with an independent conduit 222(a-n) between each testing chamber and CAI module 206. In an alternate embodiment, CAI module 206 can be coupled to the array of testing chambers through a single conduit 222. One skilled in the art will understand that other configurations can also be employed.

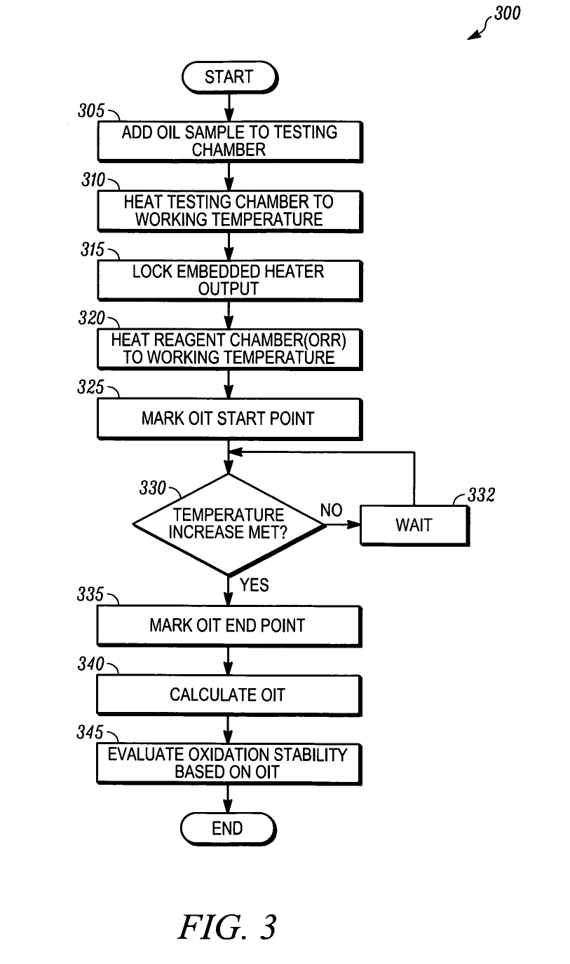

FIG. 3 illustrates a flow diagram 300 that depicts logical operational steps of a method for thermal analysis of oil oxidation stability. As indicated at block 305, the process begins, wherein an oil sample is added to a testing chamber. This operation can be performed by, for example, adding oil 214 to testing chamber 212 of FIG. 2. As indicated next at block 310, the testing chamber is heated to a working temperature. The operation depicted at block 310 can be performed by, for example, first heater 220 of FIG. 2.

As described thereafter at block 315, the embedded heater output is locked. This operation can be performed by, for example, CAI module 206 maintaining at a constant heat output first heater 220 FIG. 2. Next, as illustrated at block 320, an oxygen-releasing reagent (ORR) chamber is heated to a second working temperature. The operation depicted at block 320 can be performed by, for example, second heater 236 of FIG. 2.

As indicated thereafter at block 325, a first OIT start point is marked at a pre-determined ORR chamber temperature. The operation depicted at block 325 can be performed by, for example, CAI module 206 of FIG. 2. In an alternate embodiment, the first OIT start point is marked at the beginning of step 320, wherein the ORR chamber is heated. In an alternate embodiment, the first OIT start point is marked at the first arrival of oxygen flux into the testing chamber. As described above, other configurations can also be employed.

Next, as depicted at decisional block 330, a determination is made whether the testing chamber is at a pre-determined temperature increase. The operation depicted at block 330 can be performed by, for example, CAI module 206 and/or first heater 220 of FIG. 2. If at decisional block 330, the determination is made that the testing chamber is not at a pre-determined temperature increase, the system waits, as indicated at block 332, returning to decisional block 330. If at decisional block 330, the determination is made that the testing chamber is at a pre-determined temperature increase, the process continues to the operation depicted at block 335.

As depicted next at block 335, an OIT end point is marked at the pre-determined temperature increase. The operation depicted at block 335 can be performed by, for example, CAI module 206 and/or first heater 220 of FIG. 2. Thereafter, as indicated at block 340, an OIT is calculated based on the OIT start point and the OIT end point. The operation depicted at block 340 can be performed by, for example, CAI module 206 of FIG. 2.

Next, as depicted at block 345, the oxidation stability of the oil sample is evaluated based on the calculated OIT. The operation depicted at block 345 can be performed by, for example, CAI module 206 of FIG. 2. In one embodiment, an oxidation stability value is calculated based on the calculated OIT. In an alternate embodiment, CAI module 206 includes indicator lights and/or other visual systems that represent the oil quality as a function of the oxidation stability. For example, CAI module 206 can be configured to display a green light if the oxidation stability indicates that the sampled oil need not yet be replaced, and a red light if the oxidation stability indicates that the sampled oil should be replaced, in accordance with a pre-determined oil management plan. One skilled in the art will understand other configurations can also be employed.

Accordingly, the embodiments provide for a system, apparatus, and method for improved measurement of oil quality through thermal analysis of oil oxidation stability. In particular, stable microfabricated devices, such as, for example, a microbrick and/or microbridge, are configured for oil oxidation stability monitoring in a novel, in-line sensor configuration. Thus, improved accuracy and reliability in sensing and measurement of oil oxidation stability are provided.

It will be appreciated that variations of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

What is claimed is:

1. A sensor apparatus, comprising:
a testing chamber configured to receive a sample of oil to be tested;
a first embedded heater coupled to the testing chamber and configured to supply heat to the sample;
an oxygen flux chamber coupled to the testing chamber and configured to provide a substantially stable oxygen flux to the testing chamber; and
a control module coupled to the testing chamber, the first embedded heater, and the oxygen flux chamber, and configured to:
monitor the temperature of the sample;
direct the first embedded heater to supply heat to the sample;
direct the oxygen flux chamber to provide oxygen flux to the testing chamber;
measure an oxygen induction time (OIT); and
evaluate the oxidation stability of the sample based on the OIT.

2. The apparatus of claim 1, wherein the oxygen flux chamber comprises oxygen releasing agents and a second embedded heater.

3. The apparatus of claim 2, wherein the second embedded heater comprises a microheater.

4. The apparatus of claim 1, wherein the first embedded heater comprises a heater and a temperature sensor.

5. The apparatus of claim 1, wherein the first embedded heater and the testing chamber together comprise a disposable module.

6. The apparatus of claim 1, wherein the first embedded heater, the testing chamber, and the oxygen flux chamber together comprise a disposable module.

7. The apparatus of claim 1, wherein the control module is further configured to:
direct the first embedded heater to supply sufficient heat to the sample to maintain the sample at a predetermined first temperature;
mark a first time at a start of providing oxygen flux to the testing chamber;
mark a second time at a determination that the sample is at a predetermined second temperature; and
measure the OIT based on the first time and the second time.

8. The apparatus of claim 7, wherein the start of providing oxygen flux comprises heating oxygen releasing reagents.

9. The apparatus of claim 1, wherein the testing chamber comprises an array of testing chambers, comprising:
a plurality of testing chambers, each testing chamber configured to receive a sample of oil to be tested; and
a plurality of embedded heaters coupled to the plurality of testing chambers such that each testing chamber is coupled to at least one embedded heater.

10. A method for thermal analysis of oil oxidation stability, comprising:
providing a sample of oil to be tested to a testing chamber
heating the sample, by a first embedded heater, to a first temperature;
maintaining the output of the first embedded heater at a level sufficient to maintain the sample at the first temperature;
providing stable oxygen flux into the testing chamber beginning at a first time;
measuring a temperature increase of the sample;
upon a determination that the sample is at a second temperature, calculating an oxidation induction time (OIT) based on the time elapsed between the first time and the time the sample is determined to be at the second temperature; and
determining the oil oxidation stability based on the OIT.

11. The method of claim 10, wherein the step of providing stable oxygen flux comprises providing oxygen releasing agents to a secondary chamber coupled to the testing chamber.

12. The method of claim 11, wherein the secondary chamber is heated by a second embedded heater.

13. The method of claim 12, wherein the second embedded heater comprises a microheater.

14. The method of claim 11, wherein the first time is based on the time the secondary chamber reaches a third temperature.

15. The method of claim 10, wherein the first embedded heater comprises a heater and temperature sensor.

16. The method of claim 10, wherein the first embedded heater and the testing chamber together comprise a disposable module.

17. The method of claim 10, wherein the first embedded heater, the testing chamber, and the oxygen flux chamber together comprise a disposable module.

18. The method of claim 10, further comprising providing a plurality of oil samples to the testing chamber, wherein the testing chamber comprises an array of testing chambers, the array comprising:
a plurality of testing chambers, each testing chamber configured to receive a sample of oil to be tested; and
a plurality of embedded heaters coupled to the plurality of testing chambers such that each testing chamber is coupled to at least one embedded heater.

19. A system, comprising:
a testing chamber configured to receive a sample of oil to be tested;
a first embedded microheater coupled to the testing chamber and configured to supply heat to the sample to maintain the first sample at a predetermined first temperature;
an oxygen flux chamber coupled to the testing chamber and configured to supply a substantially stable oxygen flux to the testing chamber, comprising a second embedded microheater and oxygen releasing reagents; and
a control module coupled to the testing chamber and the oxygen flux chamber and configured to measure the temperature of the sample, to mark a first time at a beginning of stable oxygen flux from the oxygen flux chamber to the testing chamber, to mark a second time at a determination that the sample is at a predetermine second temperature, and to measure an oxygen induction time (OIT) based on the first time and the second time.

20. The system of claim 19, wherein the testing chamber comprises a testing chamber array.
